(19) United States
(12) Patent Application Publication
(10) Pub. No.: US 2025/0356364 A1
(43) Pub. Date: Nov. 20, 2025

(54) METHOD AND SYSTEM FOR INTEGRATING AGGREGATE EMISSION DATA INTO A CARBON TRADING SYSTEM

(71) Applicant: 3DATX Corporation, Amherst, NY (US)

(72) Inventors: David W. MILLER, Clarence Center, NY (US); John SCHNIER, Clarence, NY (US); Peter FUSARO, Amherst, NY (US)

(21) Appl. No.: 18/881,365

(22) PCT Filed: Jul. 5, 2023
(86) PCT No.: PCT/US2023/026897

§ 371 Date: Jan. 6, 2025

Related U.S. Application Data
(60) Provisional application No. 63/358,505, filed on Jul. 5, 2022.

(51) Int. Cl. G06Q 30/01; G06Q 40/04

(52) U.S. Cl. CPC G06Q 30/01; G06Q 40/049

(57) ABSTRACT

One or more emissions data sets from an environmental micro-device are normalized and binned based on at least one criteria filter. One or more data groups are determined from the binned one or more data sets. A first data centroid for a first portion of the one or more data groups is determined. A portion of the one or more data sets is added to the one or more data groups and a second data centroid for a second portion of the one or more data groups is determined. A credit representing a decrease between the second data centroid and the first data centroid for the one or more data groups is then determined.

### CROSS REFERENCE TO RELATED APPLICATIONS

This application claims priority to U.S. Provisional Application No. 63/358,505, filed on Jul. 5, 2022, the disclosure of which is incorporated herein by reference.

### FIELD OF THE DISCLOSURE

The present disclosure relates generally to the field of creating and trading carbon credits.

### BACKGROUND OF THE DISCLOSURE

Gaseous and particulate emissions can have an adverse effect on the environment, having the potential, depending on level of exposure and composition, to be both profoundly harmful to human health and detrimental to ecosystems and man-made infrastructure alike. As a result, many industries face ever increasing pressure to monitor, reduce, and/or limit certain emissions generated by internal combustion engines, stacks, other systems that generate emissions, or other sources.

Vehicle and transportation sector-related emissions continue to be a leading source of greenhouse gas (GHG) and air pollution in urban areas around the globe. As an example, there were over 279 million vehicles in the United States in 2019 that emitted 33% (1,750 million metric tons) of total U.S. CO₂ emissions. In the same year, the U.S. transportation sector share of total U.S. emissions for CO, NOx, and particulate matter (PM) were 54%, 59%, and 8%, respectively. Therefore, resources continue to be focused on emission reduction tactics which typically fall into two categories: current fleet inventory upgrade (e.g., roadside and/or engine bay inspection and maintenance (I/M) programs, aftermarket engine/vehicle/fuel programs, etc.) or new vehicle manufacturing (e.g., revisions of standards for newly manufactured vehicles, etc.).

In recent years, decarbonization has become a major aspect of global emissions control and reduction. Decarbonization is the process in which carbon dioxide emissions are reduced through the use of low carbon power sources. The term stems from the Paris Agreement in which dates and target goals were provided, with the ultimate goal of net zero greenhouse gas emissions by 2050. Notable achievements of the decarbonization movement include the Air Pollution Control Act of 1955, formation of the EPA, the Kyoto Protocol, and the birth of carbon trading systems.

Carbon trading systems most commonly refer to cap and trade (CAT) programs, where a central authority sets a limit (“cap”) on specific pollutants. In this system, polluters that exceed the set cap may buy tradeable credits from entities that have successfully reduced their emissions below the cap, generating a saleable surplus. CAT programs have proven extremely successful in history, with some notable programs including the U.S. acid rain program which reduced SO₂ emissions from 1980 to 2007 by 50 percent, or the U.S. EPA budget trading program which reduced ozone season NOx emissions from 2003 to 2008 by 43 percent. For some emission sources, the European Union utilizes an emissions trading approach which reduced capped emissions by 29 percent from 2005 to 2018.

Current issues with large scale CAT programs stem internationally where, regardless of international climate agreements, international boundaries create drastic differences in pollution measurement and analysis criteria. Additionally, there exists no way to consistently and to repeatedly verify the emission data across different sources. Even on smaller scales across, for example, the U.S. or its states, there exists too many different methods of emission data analysis and credit creation, and none have been shown to be especially verifiable, stable, and repeatable in large scale test programs. Additionally, most CAT programs struggle to identify a reliable absolute frame of reference for the data collective. Without such a three-dimensional data positioning, all data contributing to a carbon credit in a decarbonization strategy becomes suspect and may be discounted in the schema.

There exists a range of monitoring technologies available to measure the involved pollutants, and there are also multiple methods for data analysis/conversion of such systems into tradeable credits, but these are inaccurate, untrustworthy, and have few common aspects. This makes implementation of such different data into a traditional carbon trading system nearly impossible. Accurate and legally-defensible data comparison and verification standards are typically nonexistent and fail to faithfully replicate actual “emission events” over a period of time. What is needed is an aggregate data analysis and conversion approach that can be applied on a broader basis and allows better benchmarking of pre-existing methods for emissions data collection, such as for emissions data use in a carbon trading system.

### SUMMARY OF THE DISCLOSURE

The present disclosure provides for increased stability, security, standardization, verification, validation, trustworthiness, and/or integrity over pre-existing data-to-credit methods for fleet vehicles. This is accomplished by providing an absolute frame of reference and utilizing multiple points of similarly situated references and data groupings. The aggregate method uses a closed set of entities as references to ensure the accuracy of measured pollutant output over an analogous grouping and to enhance the overall value of the collective.

In an embodiment, a method of data analysis and data binning is applied to emission data to generate a representative data reference before converting it into one or more tradable credits for use in a carbon trading system or for carbon offsets for the voluntary carbon markets. The present disclosure allows the creation of stable tradeable credits on a large scale. The present method involves forming an aggregate data group and formulating a single data centroid to represent the entire group. The use of a single point to represent aggregate groups improves processing speeds and reduces the energy required to calculate and store the information while improving the stability and trustworthiness of the underlying asset, among other benefits. Without the use of aggregate groups, there is too much dissimilar data to process and no stability in the resulting credits.

Typically, stored data will include three pieces: a descriptor, the data, and an end. When there exist thousands to millions of data pieces, storage size and energy use increase while processing speed decreases. The use of aggregate data groups and a single data centroid reduces the number of descriptors, data, and end pieces. Therefore, the present method reduces the required storage size and energy use while increasing the processing speed.

The aggregate data groups and corresponding data centroids reduce and eliminate discrepancies in the data sets, providing a more stable, trustworthy, and integral reference for a tradeable credit.

The present method also permits authentication and verification of tradeable credits, which makes them more trustworthy and valuable in trading markets.

The present disclosure provides a system including an environmental micro-device configured to generate one or more data sets from its environment and a processor. The processor may be configured to receive the one or more data sets from the environmental micro-device, normalize the one or more data sets, bin the one or more data sets based on at least one criteria filter, determine one or more data groups from the one or more data sets after the binning, determine a first data centroid for a first portion of the one or more data groups, add a portion of the one or more data sets to the one or more data groups, determine a second data centroid for a second portion of the one or more data groups, and determine a credit representing a decrease between the second data centroid and the first data centroid for the one or more data groups.

According to an embodiment of the present disclosure, the environmental micro-device may include at least one sensor. The sensor may be a non-dispersive infrared (NDIR) sensor, a flame-ionization detector (FID) sensor, a diffusion charger sensor, a laser-light scattering sensor, an opacity sensor, an electrochemical sensor, and/or an optical sensor.

According to an embodiment of the present disclosure, the environmental micro-device may include at least one detector. The detector may be a continuous particle counter (CPC) detector and/or a quantum cascade laser infrared spectroscopy (QCL-IR) detector.

According to an embodiment of the present disclosure, the one or more data sets may include one or more of inputs, outputs, events, weather, location, time, VIN number, engine type, greenhouse gases, or criteria pollutants.

According to an embodiment of the present disclosure, the processor may be configured to verify one or more of the one or more data sets.

According to an embodiment of the present disclosure, the processor may be configured to store the credit on a distributed ledger or on an encrypted network.

According to an embodiment of the present disclosure, the binning of one or more data sets may be based on one or more of make, model, year, mileage, engine, fuel source, class, intended usage, or weight.

According to an embodiment of the present disclosure, the one or more data sets may be emissions data.

According to an embodiment of the present disclosure, the processor may include one or more processors.

The present disclosure further provides a method including receiving one or more data sets from an environmental micro-device at a processor, normalizing the one or more data sets using the processor, binning the one or more data sets based on at least one criteria filter using the processor, determining one or more data groups from the one or more data sets after the binning using the processor, determining a first data centroid for a first portion of the one or more data groups using the processor, adding a portion of the one or more data sets to the one or more data groups using the processor, determining a second data centroid for a second portion of the one or more data groups using the processor, and determining, using the processor, a credit representing a decrease between the second data centroid and the first data centroid for the one or more data groups.

According to an embodiment of the present disclosure, a non-transitory computer readable medium may store a program configured to instruct the processor to execute the method.

According to an embodiment of the present disclosure, the method may further include verifying one or more of the one or more of the data sets using the processor.

According to an embodiment of the present disclosure, the method may further include collecting the one or more data sets using the environmental micro-device.

According to an embodiment of the present disclosure, the one or more data sets may include one or more of inputs, outputs, events, weather, location, time, VIN number, engine type, greenhouse gases, or criteria pollutants.

According to an embodiment of the present disclosure, the method may further include storing the credit on a distributed ledger or on an encrypted network.

According to an embodiment of the present disclosure, the binning may be based on one or more of make, model, year, mileage, engine, fuel source, class, intended usage, or weight.

According to an embodiment of the present disclosure, the one or more data sets may be emissions data.

According to an embodiment of the present disclosure the processor may include one or more processors.

### BRIEF DESCRIPTION OF THE FIGURES

For a fuller understanding of the nature and objects of the disclosure, reference should be made to the following detailed description taken in conjunction with the accompanying figures.

FIG. 1 displays atypical pipeline emissions breakdown of petrol vehicles.

FIG. 2 displays atypical tailpipe emissions breakdown of diesel vehicles.

FIG. 3 is a flow diagram illustrating the present method and system.

FIG. 4 is a flow diagram illustrating the data binning process.

FIGS. 5A and 5B display graphical illustrations of the present method.

FIGS. 6A and 6B display graphical illustrations of the present method.

FIG. 7 is a diagram illustrating a data centroid extrapolation method.

FIG. 8 is a diagram illustrating an exemplary hierarchy of data flow from measurement to modeling of the disclosure disclosed herein, as explained in Example 1.

FIG. 9 is a diagram of the two innermost circles of FIG. 8 displaying the asset selection for detailed benchmarking of emissions and activity, as explained in Example 1.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, process step, and electronic changes may be made without departing from the scope of the disclosure. Accordingly, the scope of the disclosure is defined only by reference to the appended claims.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

The steps of the method described in the various embodiments and examples disclosed herein are sufficient to carry out the methods of the present disclosure. Thus, in an embodiment, the method consists essentially of a combination of the steps of the methods disclosed herein. In another embodiment, the method consists of such steps.

With parenthetical reference to the corresponding parts, portions or surfaces of the disclosed embodiment, merely for the purposes of illustration and not by way of limitation, the present disclosure provides an improved method for providing a tradeable credit comprising an environmental micro-device, a data set, one or more verification steps, normalization, binning the data sets into smaller aggregate data groups using one or more bin filters, creating a single data centroid for the aggregate data group, and generating a tradeable credit representing the aggregate data group which can optionally be stored in blockchain or another encrypted network. The present disclosure provides for increased stability, security, standardization, trustworthiness, and/or integrity over pre-existing credit creation methods.

An embodiment of the method may further include compiling “unbinnable” or unverifiable data sets and generating a tradeable credit of lesser value than that of a properly verified and binned aggregate tradeable credit. Even further, the method may include marketing and monetizing the aggregate tradeable credit after a viable methodology is created and verified. This includes buying or selling the aggregate tradeable credit in a trading-based market.

The present disclosure further provides a system including a processor that is configured to receive a plurality of data sets from an environmental micro-device. The environmental micro-device may be a portable or stationary device that generates information or data from its environment. For example, the environmental micro-device may generate data from one or more sensors or an operator input. The environmental micro-device may be installed or used in conjunction with a pre-existing system, such as with a vehicle. The environmental micro-device may be in electronic communication with a processor (such as a PC or a server) that can provide additional calculations based on the measurements.

The environmental micro-device may contain emissions sensors and/or detectors known in the industry, such as non-dispersive infrared (NDIR), flame-ionization detector (FID), diffusion charger, laser-light scattering, opacity, electrochemical, non-dispersive ultra-violet (NDUV), diffusion charger, continuous particle counter (CPC), quantum cascade laser infrared spectroscopy, infrared laser absorption modulation, and/or optical sensors. The environmental micro-device also may include sensors to determine one or more of temperature, humidity, pressure, location, etc. The environmental micro-device may be remotely controlled and operated. One or more of the same or different environmental micro-devices may be used in conjunction to provide the data sets.

A data set can be a collection of information. The contents of the data set depend on the implementation of the environmental micro-device. The data set may include, for example, inputs, outputs, events, weather, location, time, etc. With respect to vehicles and emissions, the data set may include information relating to VIN number, engine type, greenhouse gases, criteria pollutants, etc. These data can be values in a spreadsheet or database, for example.

The collection process of a data set and the data set itself may be verified. Verification can make the resulting end credit more valuable than a credit that was not verified. Collection verification may include, for example, utilizing on-board sensors to ensure the implementation of the environmental micro-device was performed as defined by, for example, a governmental body. The verification may also be performed by one or more representative persons of the governmental body. The data set may be verified (e.g., scanned by a computer program or artificial intelligence) to ensure that the data is, for example, not corrupted, altered, or missing components. The verification process may be done by a third party. The verification process may be regulated by an overriding entity.

Verification may include the use of a management-by-exception software to identify details or events that fall outside of a pre-defined set of parameters, a blockchain and/or a chain-of-custody software security, and other measures built-in to ensure that human operators and decision makers are provided with accurate information.

Normalization allows data sets from various sources to be comparable. The data set may be normalized by applying one or more correction factors, formulas, calculations, or running a computer program, etc. For example, different vehicles have different driving modes, and even identical vehicles or mobile sources can have different emissions profiles dependent upon geographical area, registry, lifetime use, fuel quality, etc. Normalization can use an external absolute X and Y axis, and eliminates extraneous outlier data, in order to compare and value seemingly dissimilar data sets.

Furthermore, the data set may be binned based on certain criteria. The binning can be based on information pertaining to the source environment. For example, binning may be based on the source, location, time, etc. Regarding vehicles, the binning can be based on, for example, make, model, year, mileage, engine, fuel source, class, intended usage, weight, etc.

A data group may be a compilation of data sets. In an embodiment, the data groups may be aggregated and include different, but similar, data sets.

Binning of the data sets may result in any number of different aggregate data groups. Each data set in the same aggregate data group is generally similar due to the normalization and binning process. Data binning may occur several times to increasingly refine the raw data sets. The more filtered an aggregate data group is, the better each individual data set in the data group will correlate. Thus, a single statistical representation of the aggregate data group will better represent all the individual data sets. The multiple binning or filtering process can be performed in series or in parallel. In series, the data set is analyzed using one filter at a time. In parallel, the data is analyzed using multiple filters at one time.

Binning allows for the evaluation of a wide variety of mobile sources, where each mobile source may have unique data that is specific to its own category. The data binning process identifies the similar data sets, assigns a value, and identifies and values dissimilar data sets in differing percentages.

The aggregate data groups may be represented by a data centroid. The data centroid is the mathematical “center” of the contents of the aggregate data group. In an example, an aggregate data group containing from one to one-thousand data sets can be represented by one data centroid. The data centroid could be calculated by, for example, a weighted formula, total over a time period, average, rate, mean, mode, or an advanced mathematical model. Regarding vehicle emissions, the data centroid may represent, for example, the total CO₂ equivalent emissions during one year for the vehicles included in the aggregate data group.

A data set may be binned into an aggregate data group that already has a data centroid. The data centroid can be continuously re-calculated when a new data set is binned into the underlying aggregate data group. The aggregate data groups may continuously grow over time; and therefore, the data centroid may become more stable and stronger with time. A stopping criterion may exist to limit the size of the aggregate data groups. The stopping criteria could be based on the value of the group, size, time of existence, etc.

Data sets may be re-inputted to the method and system if, for example, there was an intentional and substantial change made to the environment of the environmental micro-device. In an example, the first data set may be with the environmental micro-device near a vehicle with an outdated catalytic converter and the second “repeat” data set may be with the environmental micro-device near the same vehicle with an updated catalytic converter.

One of the binning filters may include checking if the data set is a repeat, as a way to prevent the elimination of redundant data sets. If it is repeated, it is binned into a subset of the previous data group. In an example, the first data set may go into “aggregate data group Ta” and the corresponding repeat data set may go into “aggregate data group 1b”.

The determination of if the data set is a repeat can be performed by computer program, artificial intelligence, or manually by an operator. There may exist bin filters or criteria to determine if a data set is repeated. Regarding vehicles, the criteria may include, for example, the vehicle VIN number. In an example, if the VIN number is already pre-existing within the data sets, then the new data set with the same VIN number is a repeat data set.

An aggregate data group containing the repeat subsets may have a data centroid calculated in the same way as the original aggregate data group. When compared to the original data centroid, the repeat data centroid can, in linear terms, increase or decrease from the original. In the event of a decrease, the difference between the two data centroids can be converted into a tradeable credit. An aggregate data group and its repeat subset do not need to have the same number of internal data sets in order to generate a credit. For example, a weighted average can be used which accounts for the different group sizes.

The decrease is determined by the emissions plus mass flow over time which provides a grams per x value. The reduction differential (change) data is subtracted from the baseline data (starting point) and these reductions, now expressed in a measure of weight over a certain period are assigned a monetary value based on the carbon registry being used.

Each decrease in data centroids can be represented by one or more tradeable credits based on the defined market value of the tradeable credits at the time of creation. An increase in the data centroid may also occur. In this case, a tradeable credit is not created and may even be removed or destroyed from the trading market, if permitted. The tradeable credits may be marketed and monetized in a trading market. The tradeable credits may optionally be stored in blockchain or an encrypted network to ensure they are not altered or manipulated for their lifetime. An encrypted network may be specifically set up to protect personally sensitive data in order to have the ability to adhere to data privacy laws and regulations, such as the European Union General Data Protection Regulation (GDPR). This may be regulated by the carbon registry whose jurisdiction credit generation falls under such as the Climate Action Registry, VERRA or the Gold Standard. This can add security to the method and system. The aggregate data groups and corresponding data centroids reduce and eliminate small discrepancies in the data sets, providing a more stable, trustworthy, and integral reference for a tradeable credit.

Although disclosed with regard to a single data set input, the present method and system can be applied to batches of data sets. The batches of data sets can be, for example, for a fleet of the same vehicles. Although described with regard to vehicle emissions, the method and system can be applied to emissions relating to, for example, factories, vessels, locomotives, aircraft, highways, cities, forests, etc.

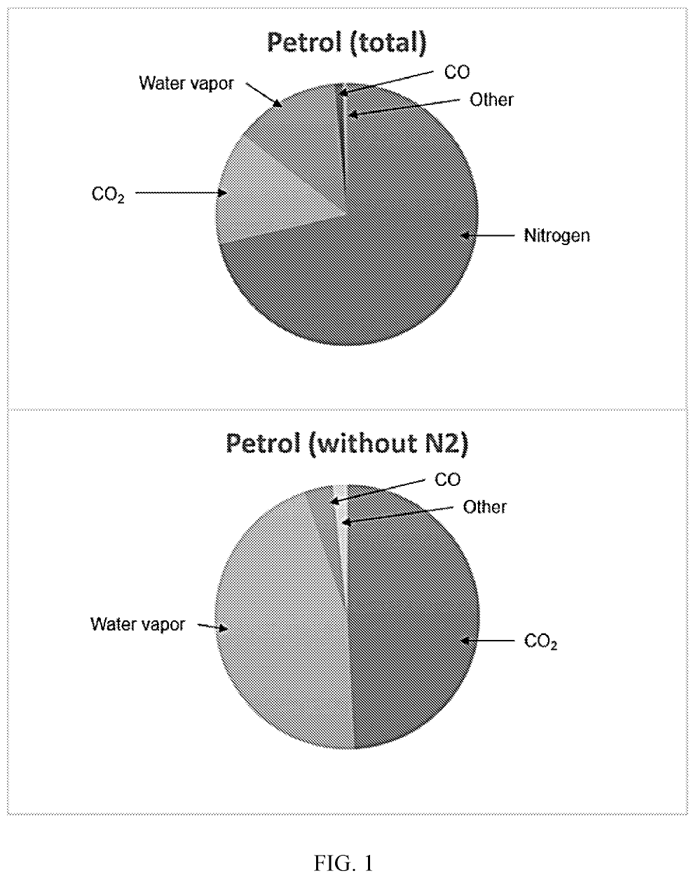

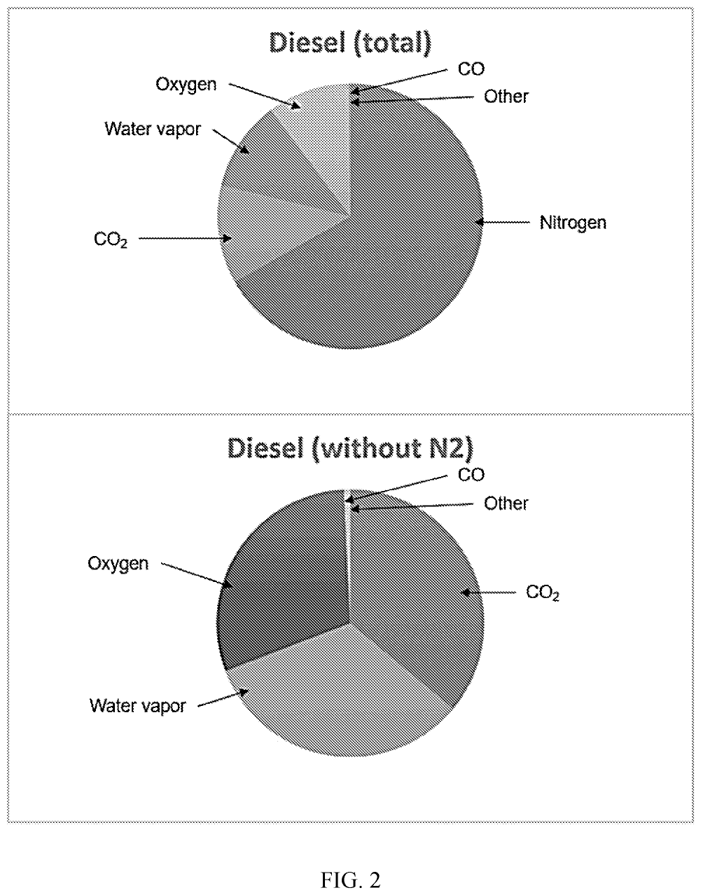

FIGS. 1 and 2 show the typical tailpipe emissions breakdown of petrol and diesel vehicles, respectively. For petrol vehicles, typical tailpipe emissions consist of 71 percent nitrogen, 14 percent carbon dioxide, 13 percent water vapor, 1 percent carbon monoxide, 0 percent oxygen, and 0.5 percent other pollutants consisting of nitrogen oxides, hydrocarbons, sulfur dioxide, and particulate matter.

FIG. 2 shows the typical tailpipe emissions breakdown for diesel vehicles of 67 percent nitrogen, 12 percent carbon dioxide, 11 percent water vapor, 0.045 percent carbon monoxide, 10 percent oxygen, and 0.255 percent other pollutants consisting of nitrogen oxides, hydrocarbons, sulfur dioxide, and particulate matter.

In either case, when nitrogen, which is a non-toxic inert gas, is removed from the breakdown, carbon dioxide becomes the major emission. Although all tailpipe pollutants play a role in global warming, carbon dioxide has the largest impact, mostly due to it having the greatest emission volume globally.

Due to the increased impact from carbon dioxide, carbon credits are developed as carbon dioxide equivalent. Carbon dioxide equivalent, abbreviated as CO₂-eq is a metric used to compare the emission of various greenhouse gases based on their global-warming potential, by converting amounts of other gases to the equivalent amount of carbon dioxide with the same global warming potential.

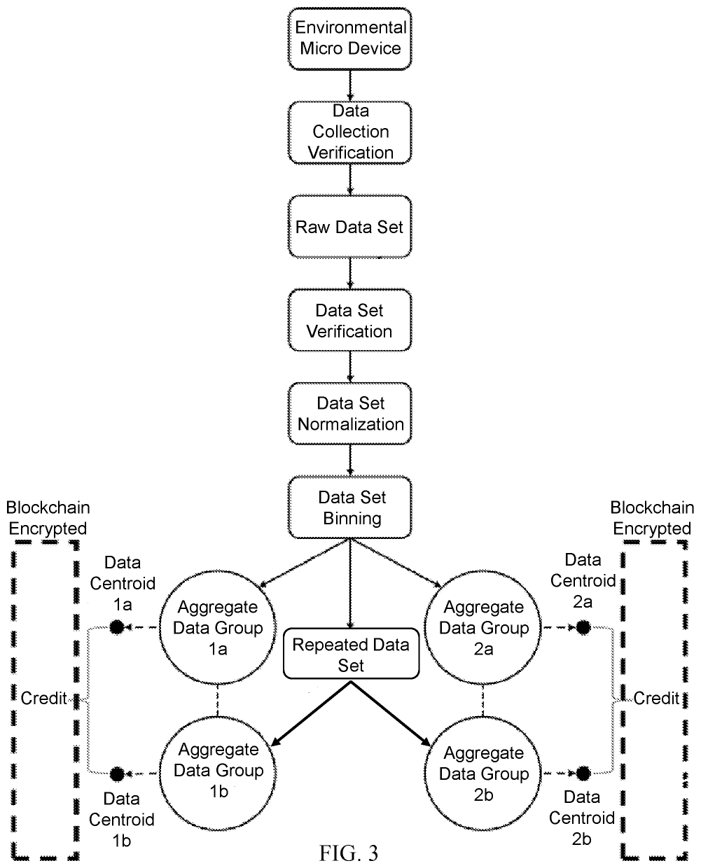

For example, one ton of methane is equal to 25 tons of CO₂-eq because it has a global warming potential 25 times that of CO₂. As shown in FIG. 3, the present method comprises the steps and components of an environmental micro-device, data collection verification, one or more raw data sets, data set verification, data set normalization, data set binning. This leads to the creation of aggregate data groups, a data centroid calculation for each aggregate group, and a tradeable credit creation based on the difference between the data centroids of repeated aggregate groups. The final credit can be stored in blockchain or another encrypted network for security.

The data set may be normalized by applying one or more correction factors, formulas, calculations, or running a computer program, for example. The data set normalization may be performed to make data sets from different sources comparable. The data set can be trimmed so that only the most relevant information is saved and used to increase processing speed and reduce energy requirements.

Tradeable credits in a carbon trading system are typically based on CO₂ equivalence, which is the representative equivalence of the data (i.e., pollutants) in terms of CO₂ emissions, based on the global warming potential. Thus, the data set normalization may be performed to convert to CO₂ equivalence or otherwise make different sources comparable.

Regarding vehicle emissions, the normalization process may include a variety of CO₂ equivalence factors used to convert data into the representative equivalent of CO₂ emissions.

In an example, if the data relates to a non-emissive vehicle, such as an electric vehicle, energy use may be converted to CO₂ equivalence using the following factor.

$$884.2 lbs {CO}_{2} / MWh \times 1 metric ton / 2 , 204.6 lbs \times 1 / ( 1 - 0.073 ) MWh delivered / MWh generated \times 1 MWh / 1 , 000 kWh \times = 4.33 \times 10 - 4 metric tons {CO}_{2} / kWh$$

In another example, if the data is in the form of gallons of fuel (i.e., gasoline or diesel), then the following factors may be used for normalization, respectfully.

$$8 , 887 grams of {CO}_{2} / gallon of gasoline = 8.887 \times 10 - 3 metric tons {CO}_{2} / gallon of gasoline$$

$$10 , 180 grams of {CO}_{2} / gallon of diesel = 10.18 \times 10 - 3 metric tons {CO}_{2} / gallon of diesel$$

The data normalization allows all data sets to be represented with respect to the same reference (i.e., CO₂ equivalence) before the binning process.

In a further example, normalization may also include equating the time periods of data sets. For example, a data set from an environmental micro-device may include hours of data but must be extrapolated to the entire year or other time span that the tradeable credit represents.

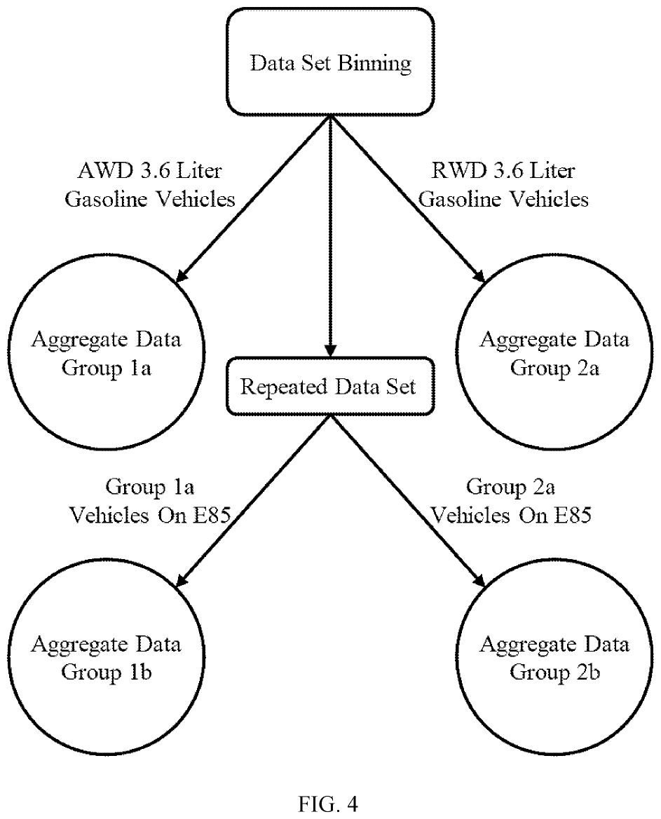

Furthermore, the data set may be binned based on its contents. FIG. 4 is an illustration of the binning process. The process of binning includes sorting the normalized data sets based on pre-determined filters. The bin filters can be based on information pertaining to the environment. For example, binning may be based on the source, location, time, etc.

Regarding vehicles, the binning can be based on, for example, make, model, year, mileage, engine, fuel source, class, intended usage, weight, etc.

The data sets can be collected over different distances and different time periods, which is normalized prior to binning.

In FIG. 4, example bin filters of (all-wheel drive) AWD 3.6-liter gasoline and rear-wheel drive (RWD) 3.6-liter gasoline vehicles are used. In an example, corresponding data sets would be sorted into “aggregate data group 1a” and “aggregate data group 2a”, respectively. Although two aggregate data groups are used as an example, binning of the data sets can result in any number of different aggregate data groups. Further, in an embodiment, there may be a bin allocated for outliers. In the example of FIG. 4, a third bin could be added for data sets that cannot be sorted into either aggregate data group 1a, 1b, 2a, or 2b. In an example, a data set relating to AWD 3.6-liter diesel powered vehicles could be sorted into the third bin, 3a.

An outlier bin may also be used to store data sets that meet the required criteria but were unverifiable either during the data collection verification or the data set verification steps. For example, an aggregate data group 1c and 2c could be added for unverifiable data sets that otherwise meet the binning criteria of 1a and 2a. These unverified data groups could be used to generate a tradeable credit of lesser value than that of 1a and 2a. For example, if a data set is missing data and therefore cannot be verified, it may be used to represent a lesser value tradeable credit.

The aggregate data groups may be resorted such that data sets may interchange between groups until the tradeable credit is created and the data group is effectively locked out. Data sets may be binned more than once, which is a “series” binning method. In a series binning method, and relating to FIG. 4, aggregate data group 1a may, for example, be further binned based on the source location of the data, the age of the vehicle, etc.

Alternatively, data sets may be binned in “parallel”. In a parallel binning method, and relating to FIG. 4, the filter for the aggregate data groups may include more than one criterion. FIG. 4 represents a parallel binning method since the filters include more than one criterion: AWD and 3.6 liter engine. A data set must meet all the binning criteria to enter an aggregate data group.

Each aggregate data group has a counterpart which is denoted by a “b” in FIG. 4. The counterpart “b” bin is for repeat data sets. A repeat data set, for example, can have one or more components of the environment that was intentionally and substantially changed. In the example of FIG. 4, the data groups 1b and 2b represent the vehicles in groups 1a and 2a running on E85 fuel instead of gasoline. As a further example, the first data set may be with the environmental micro-device near a vehicle with an outdated catalytic converter is sorted into bin “a” and the second “repeat” data set may be with the environmental micro-device near the same vehicle with an updated catalytic converter, which is sorted into bin “b”. There can exist any number of counterpart/subset bins, such as from zero to one-thousand.

The aggregate groups may be modified until a stopping criterion is met. The stopping criterion can be the value of the aggregate group. A “highest reasonable value” can be assigned, which could be 80-100% of the highest value group. The stopping criterion could also be, for example, the size of the group or time existence of the group. Once the stopping criterion is met, the aggregate groups can no longer be modified and are converted into a tradeable credit. The tradeable credit and related information can be stored in blockchain or another encrypted network for security.

In an embodiment, the data centroid may represent one aggregate data group. The data centroid may a single data point, which can be in the form of, for example, total CO₂ equivalence emitted in one year. As shown in FIG. 3, aggregate data group 1a has a data centroid 1a, aggregate data group 1b has data centroid 1b, and so on. Each data centroid represents only one aggregate data group.

The data centroid can be a good representation of the individual data sets that make up an aggregate data group. As the size of the aggregate data group increases, the data centroid becomes more stable and secure. Thus, the resulting tradeable credit becomes more valuable. The data centroid can be calculated, for example, by using the following formula.

$$Data centroid = average of the aggregate data group \times sample size of the aggregate data group$$

The data centroid can be continuously updated as the aggregate data group grows in size, or it can be updated based on a time period, for example, once per year or once per seven years.

The use of an aggregate data group and corresponding data centroid removes the effects of discrepancies in the individual data sets, resulting in a more stable, valuable, and trustworthy tradeable credit.

A repeat aggregate data group is formed for each aggregate data group as new data comes in that has, in some way, been intentionally and substantially changed from a previous data set. A new data centroid is calculated for the repeat aggregate data group with the same method as for the original aggregate group.

A tradeable credit is generated if the value of the repeat data centroid is less than that of the original group's data centroid.

Since the data centroid may incorporate the size of the aggregate data group, an aggregate data group and its repeat subset do not need to have the same number of internal data sets to generate a credit. Thus, the aggregate data group and its repeat subset can different numbers of internal data sets.

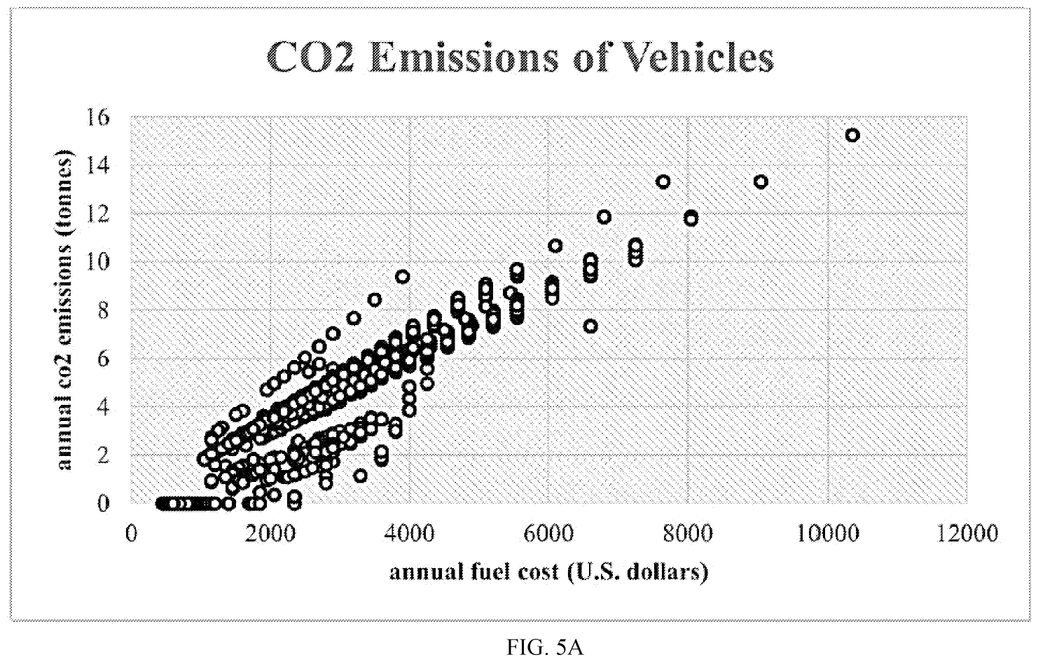

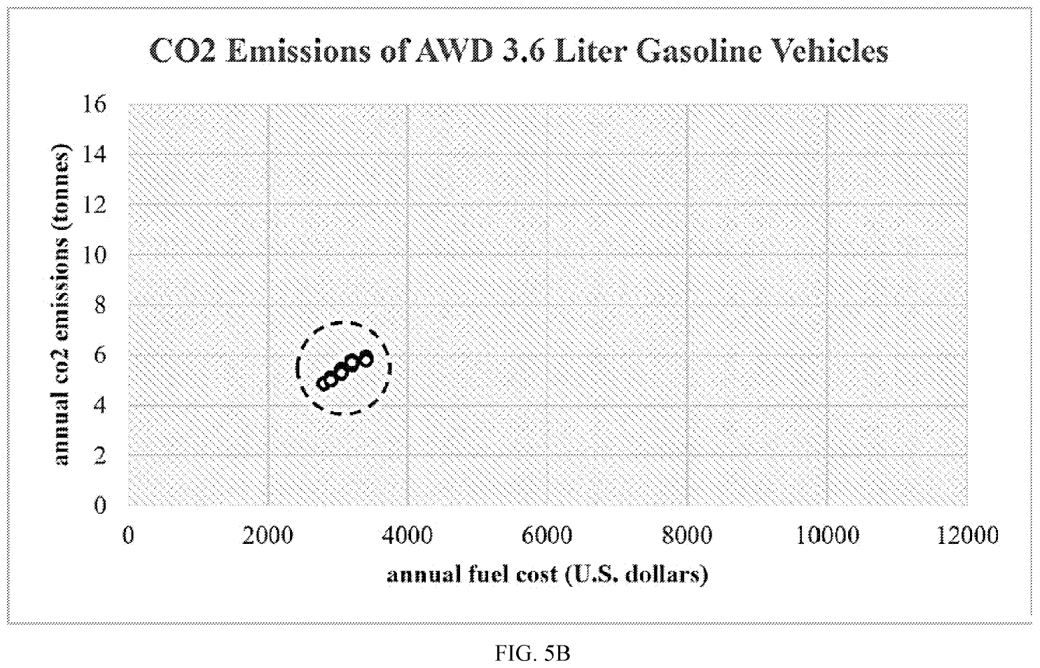

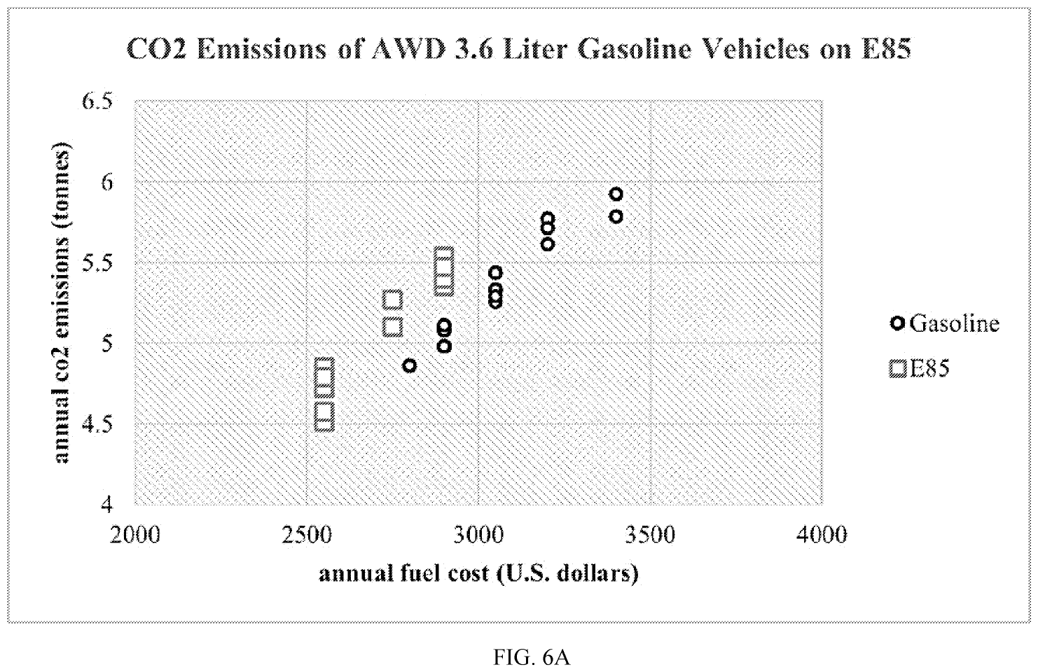

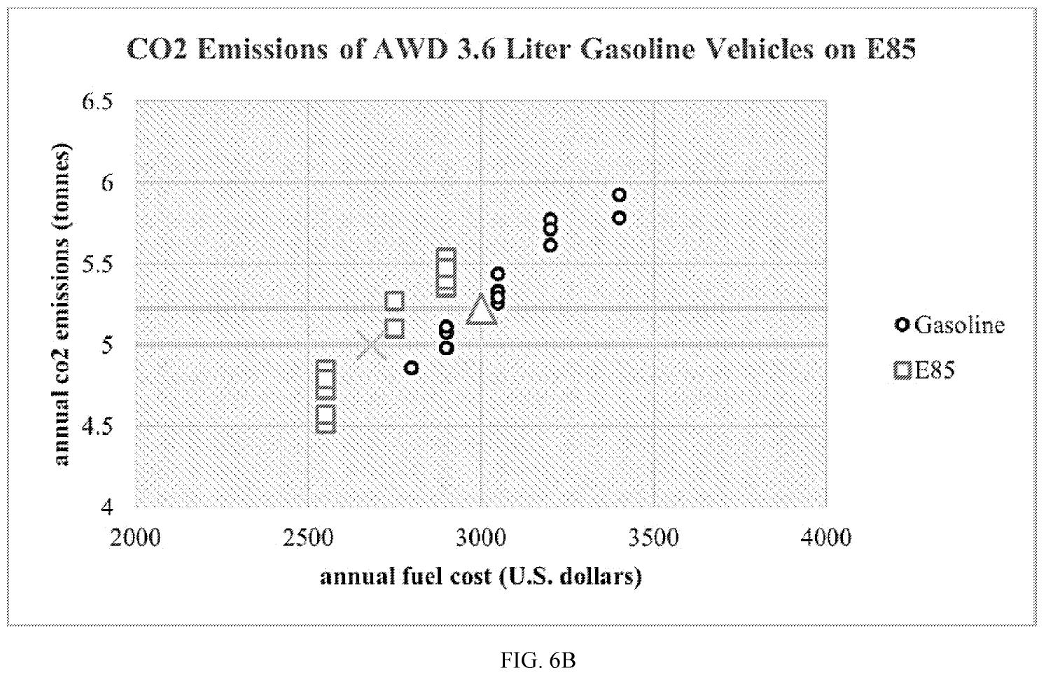

FIGS. 5A-5B and 6A-6B are illustrations of the data binning and data centroid calculation, based on actual vehicle emission data. In FIGS. 5A-5B and 6A-6B, vehicle emission data is plotted with annual fuel cost (in U.S. dollars) on the x-axis and annual CO₂ emissions (in tonnes) on the y-axis.

FIG. 5A (top) is a plot of the entire vehicle emissions data, which is over 40,000 individual data points. With this amount of data, there is no way to process it into stable tradeable credits of high value. What is needed is an aggregate data group approach.

FIG. 5B (bottom) represents an example aggregate data group, which includes AWD 3.6-liter gasoline vehicles. This data group contains approximately 30 similar data points, which is much more manageable than the entire emissions data of over 40,000 data points. Since each of the data points are binned by the same filter, they are all similar, which is shown by them existing in same area of the plot, which is shown by the dashed circle. Therefore, a single data centroid can be a good representation of all the data points inside this aggregate data group.

The data centroid changes location greatly based on the level of filtering. However, as the level of filtering is increased, the data centroid better represents that aggregate data group as the filtering is increased. The binning and data centroid process can be modeled by a limit function where a certain amount of filtering could lead to an extremely similar aggregate data group as if it were actually a single data point, which could result in lower stability. Therefore, there is an optimal level of filtering where the data centroid well represents many individual data sets while still being stable and accurate. An error function may be used to determine the optimal filtering amount.

FIG. 6A (top) shows the same aggregate data group as in FIG. 5B as well as the same vehicles when running on E85 instead of gasoline. The gasoline data group is represented by circles and the E85 data group is represented by squares. In the example of FIG. 4, these groups represent aggregate data group 1a and Tb, respectively. It can be seen that when running on E85, the data points have shifted to the left and down.

FIG. 6B (bottom) shows a data centroid for each aggregate data group. In this graphical example, the data centroid was simply calculated by taking the average x and y coordinates of the data points inside each aggregate data group. The data centroid for the gasoline data group is represented by a triangle whereas the E85 data group is represented by an x. A line that intersects each data centroid is drawn horizontally. From FIG. 6B it can be deduced that the y-value of the E85 data group is lower than that of the gasoline data group. In an embodiment, quantitatively, there was a reduction in CO₂ emissions from 5.2 tonnes per year to 5.0 tonnes per year when the vehicles switched from gasoline fuel to E85. In an embodiment, a tradeable credit representing the reduction, 0.2 tonnes, can be generated.

Further, if the going price of a CO₂ credit was $100/tonne, then the value of the reduction is $20. The entity associated with changing the vehicles from gasoline to E85 would be credited $20 worth of credits, or 0.2 credits. Once created, the credit may increase or decrease in value based on the trading market.

The value of the resulting tradeable credit may be split up among the involved parties. For example, 10% may go to the credit creation company.

Since the example used in FIGS. 5 and 6 were represented graphically, a simple average of the x and y coordinates was used to determine the data centroid.

For vehicles, tradeable credits typically represent the total CO₂ equivalent emissions over one or more years. To get an exact number for the emissions amount, the emissions would have to be measured for every second that the vehicle is turned on. For obvious reasons, this is not possible or efficient. Traditional methods of finding an emissions amount include guessing and poorly represent the actual emissions amount, which leads to lesser value in tradeable credits. The present method utilizes modeling and extrapolation to generate an accurate emissions amount.

In an embodiment, a close emissions amount can be determined by using three different data sources and extrapolating the most accurate one using the other two lesser accurate sources. For example, in order of accuracy, the data sources can be exact emissions measurement from an environmental micro-device, an activity monitor, and a usage amount.

For a vehicle, the activity monitor could provide information such as distance, time, date, acceleration, speed, etc. In an example, since fuel compositions change during seasons, the time and date can be used to extrapolate the emissions amount more accurately.

If there are more environmental micro-device measurements at various activity states, then the extrapolation can be more accurate. As an example, emissions measurements during the activity states of acceleration, constant speed, and deceleration can allow a more accurate extrapolation than if the activity state was solely constant speed.

Activity data is easier to gather than actual emission data, but still may not be measurable over an entire year. Thus, a usage amount can be included to extrapolate the data further. The usage amount could be the amount of fuel burned, or distance driven, as these numbers are often already measured and easily obtainable for vehicles.

Since the extrapolation of the environmental micro-device data is performed based on measured quantities, embodiments disclosed herein provide an accurate emissions quantity and credit.

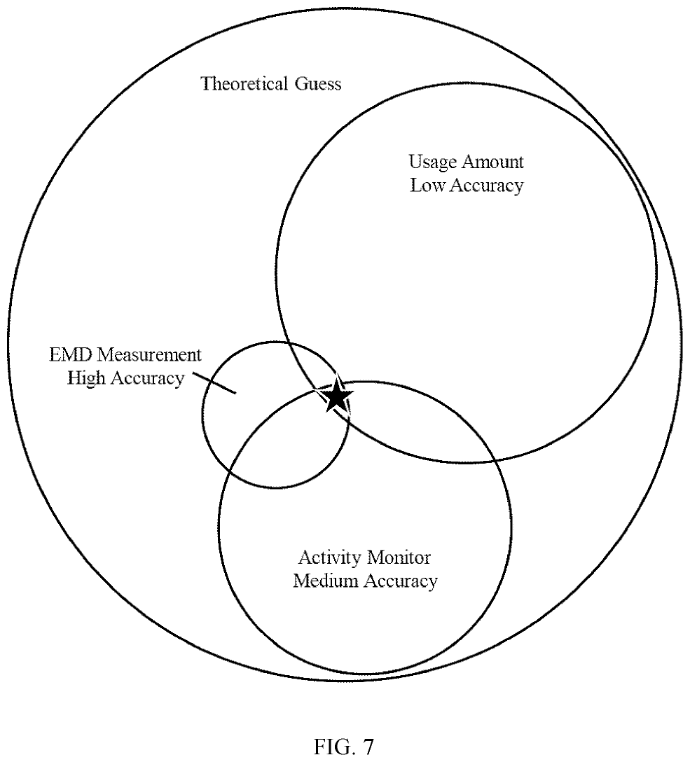

FIG. 7 is an illustration of using three data sources of various accuracies to determine an accurate emissions amount. A larger circle indicates a lesser accuracy. As shown, the order of increasing accuracy is theoretical guess, usage amount, activity monitor, and environmental micro-device measurement. The intersection of all three data sources is marked by a star, which is determined by extrapolating data from all three sources, which provides a more accurate number than a theoretical guess.

The calculation of the intersection can include weighting so that the environmental micro-device (high accuracy) data has a higher weight than the activity monitor, and so on. This way, a more accurate credit may be assigned if there is more data from an environmental micro-device. A greater number of environmental micro-device measurements could also lead to a greater emission reduction and a greater credit value.

For example, if the emissions amount is calculated based solely on the usage amount, the number would be far less accurate than if the calculation included data sets from an environmental micro-device and activity monitor. In general, a more accurate credit is more valuable even if the resulting emission reduction amount is less. Higher value credits benefit the trading market as it will not get diluted with low value/accuracy credits.

The tradeable credit value may be calculated based on the difference between a data centroid and its corresponding repeat data centroid, as well as the value of the tradeable credits at the time of creation. The tradeable credit value may be calculated, for example, using the following formula.

$$Tradeable credit value = ( data centroid - repeat data centroid ) \times value of one tradeable credit ( price / unit )$$

The number of tradeable credits may also be calculated by dividing the tradeable credit value by the going price per credit.

Credit creation may be carried out by a carbon developer which establishes a baseline methodology, measures the emissions reductions, and puts the process through an established carbon registry for validation and verification. Another independent third party can verify the methodology and credit generation before credits can be issued by the registry.

Binned aggregate data groups may optionally be stored in blockchain for security and transparency using the general ledger aspect of blockchain technology. The tradeable credit may also be stored using another encrypted network for security during the lifetime of the credit. The credit may also be stored and managed using, for example, a customer relationship management program or another data processing platform.

Measurement of the pollutants may be performed as part of a yearly vehicle checkup such as a periodic technical inspection.

While disclosed with respect to vehicle emissions, the present method and system can be applied to the emissions of factories smokestacks, vessels, locomotives, aircraft, highways, cities, and forests after being validated and verified by the registries.

It will be understood that, while exemplary features of the method herein have been described, such an arrangement is not to be construed as limiting the disclosure to such features. The method may be implemented in software, firmware, hardware, or a combination thereof. In one mode, the method is implemented in software, as an executable program, and is executed by one or more special or general purpose digital computer(s), such as a personal computer (PC; IBM-compatible, Apple-compatible, or otherwise), personal digital assistant, phone, tablet, workstation, minicomputer, or mainframe computer. The steps of the method may be implemented by a server or computer in which the software modules reside or partially reside.

Generally, in terms of hardware architecture, such a computer will include, as will be well understood by the person skilled in the art, a processor, memory, and one or more input and/or output (I/O) devices (or peripherals) that are communicatively coupled via a local interface. The local interface can be, for example, but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the other computer components.

The processor(s), i.e. of the control system, may be programmed to perform the functions of the method embodiments disclosed herein. The processor(s) is a hardware device for executing software, particularly software stored in memory. Processor(s) can be any custom made or commercially available processor, a primary processing unit (CPU), an auxiliary processor among several processors associated with a computer, a semiconductor-based microprocessor (in the form of a microchip or chip set), a macro-processor, or generally any device for executing software instructions.

Memory is associated with processor(s) and can include any one or a combination of volatile memory elements (e.g., random access memory (RAM, such as DRAM, SRAM, SDRAM, etc.)) and non-volatile memory elements (e.g., ROM, hard drive, tape, CDROM, etc.). Moreover, memory may incorporate electronic, magnetic, optical, and/or other types of storage media. Memory can have a distributed architecture where various components are situated remote from one another, but are still accessed by processor(s).

The software in memory may include one or more separate programs. The separate programs comprise ordered listings of executable instructions for implementing logical functions in order to implement the functions of the modules. In the example of heretofore described, the software in memory includes the one or more components of the method and is executable on a suitable operating system (O/S).

The present disclosure may include components provided as a source program executable program (object code), script, or any other entity comprising a set of instructions to be performed. When a source program, the program needs to be translated via a compiler, assembler, interpreter, or the like, which may or may not be included within the memory, so as to operate properly in connection with the O/S. Furthermore, a methodology implemented according to the teaching may be expressed as (a) an object-oriented programming language, which has classes of data and methods, or (b) a procedural programming language, which has routines, subroutines, and/or functions, for example but not limited to, C, C++, Pascal, Basic, Fortran, Cobol, Ped, Java, and Ada.

The following example is presented to illustrate the present disclosure. It is not intended to be limiting in any matter.

### Example 1

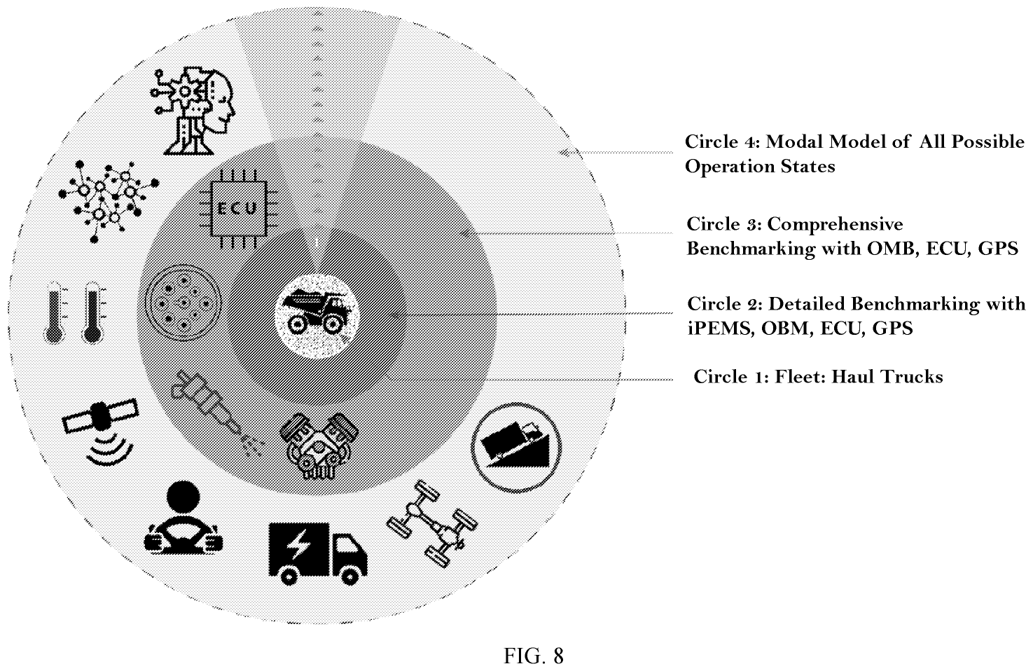

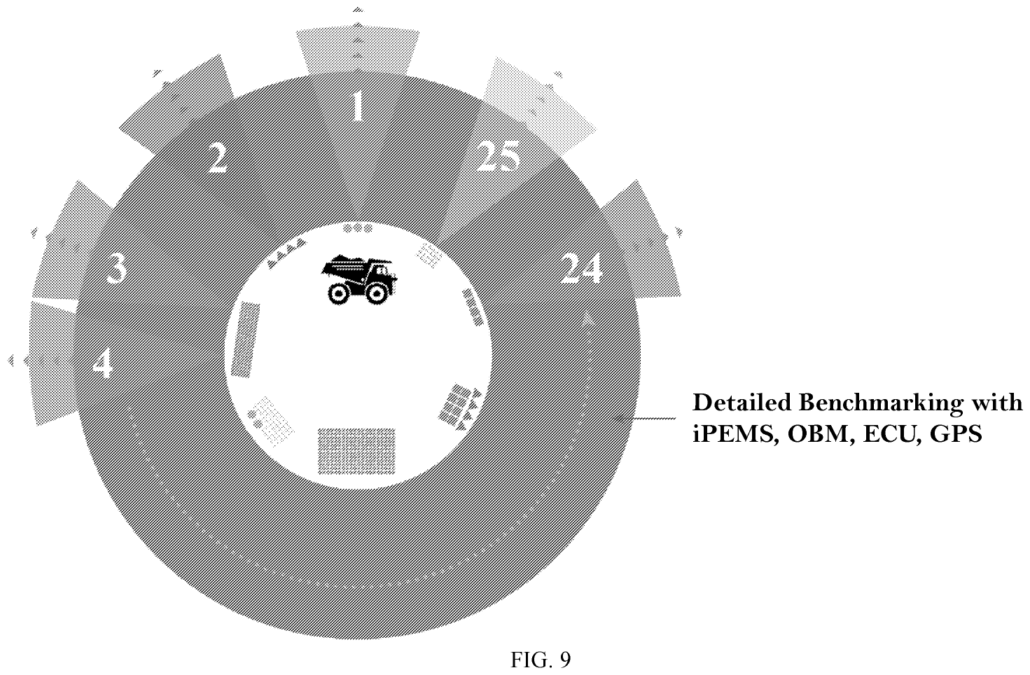

The following in an example of the hierarchy of data flow using the system and method disclosed herein. FIG. 8 displays a bird's eye view of the hierarchy of data flow, and FIG. 9 focusses on the two innermost circles for visual clarity.

In FIG. 8, the concentric circles represent specific zones of knowledge and data execution about the equipment. The innermost circle is what already exists, such as groups of vehicles or fleets. The vehicles or fleets is the source of the emissions, but the true quantity and quality of these emissions is not fully known. These emissions are represented in the increasingly larger circles which may be measured and modelled using an embodiment of the present disclosure.

Circle 1 of FIG. 8 represents an entire fleet of 650 haul trucks marked with dots in various shapes and sizes. The different shapes and sizes represent the variety in such fleet due to make and model, model year or age, emissions control devices, and duty cycle (or work performed).

Once assets to be tested are determined, detailed data collection is done to establish baseline fuel use rates and emissions rates, all recorded on second-by-second basis (1 Hz). As shown in Circle 2 of FIG. 8, high accuracy tailpipe emissions may be measured for three 8-hour work shifts using the 3DATX parSYNC FLEX integrated portable emissions measurement system (iPEMS). An embodiment of parSYNC is disclosed in U.S. Pat. No. 10,190,945, which is incorporated by reference in its entirety. The three 8-hour measurements may be conducted in separated weeks (and not back-to-back) to generate sufficient sample size for a large variety of operation modes since equipment can work at different depths or material. However, testing may be completed during any period of time, for any amount of days.

Simultaneously during this data collection period, two other measurements are conducted: (1) tailpipe emissions using onboard measurement (OBM) devices, and (2) equipment activity using electronic control unit (ECU) loggers and global positioning systems (GPS). All emissions and activity data are binned into modes of engine load, fuel use rate, exhaust temperature, etc., identified as the modal model. A final set of data may be payload (tons), nature of work performed, and any available details about the surfaces, including grade.

After this portion of data collection, a less detailed set of data may be collected for a much longer period, as depicted by the increasing area of the slice as shown in Circle 3 of FIG. 8. This data may be recorded using a subset of the wider array of instruments used in the previous stage (Circle 2). The on-board measurement (OBM), ECU, and GPS devices once installed, measure non-stop until the end of program, which means hundreds of hours of high-resolution data per equipment. All emissions and activity data are binned per the modal model.

The high-density and high-quality data from the Circle 2 is used to validate the high-resolution and high-coverage data in Circle 3. Once that correlation is established within the modal model framework, the Circle 3 data continues to inform the model and allow for powerful “what-if” scenario modelling (as explained below). Note that if OBM and ECU are continuously measured for the life of the equipment, these can be directly used to provide real-time transient, hourly, daily, monthly, or annual estimates of fuel use and emissions, and there will be no need for moving onto the Circle 4 for that specific asset.

As shown in Circle 4 of FIG. 8, the modal model uses data from Circle 2 and Circle 3 to simulate all activity modes that can possibly exist for every asset in a truck fleet. Each inner circle does an information hand-off to the next bigger circle. This results in a Circle 4 model that is robust. Circle 4 is where all the optimization options and what-if scenarios related to reductions in fuel use and/or emissions are made available. Examples of such options and scenarios include some or all of the following. What-if the fuel is switched to renewable diesel? What-if the equipment were fully electric? What-if the operator enacts less aggressive acceleration? What-if there is autonomous driving because machines drive differently than humans? What-if the grade or curvature of a ramp is changed? What-if there is reduction in non-useful-work idling periods? How can payloads be optimized between tons/trip and total trips? What are the true benefits (not OEM claims) of upgrading the fleet to newer models? These and other scenario-based questions may be answered by the data collected by Circle 2 and Circle 3 and modelled in Circle 4.

As shown, Circle 4 represents all possible mobile sources capable of being assessed for carbon credits. The centroid process allows for the focus on specific mobile sources assuming valid data exists. Circle 4 may be quantified based on the first three circles identified (Circles 1, 2, and 3), and, due to the chain-of-custody approach from one circle to the next, the method allows for the application of a defensible monetization value.

In FIG. 9, the focus is on sample selection. Groups might be based on manufacturer, model, model year, emissions control devices, operating conditions, future life in the fleet, or other groups. Some groups might be small but consequential, so at least one asset from the group may need to be instrumented with the invention disclosed herein, as demonstrated by the dots, larger triangles, and larger squares. Another group might be large and consequential, and thus requiring multiple assets to be instrumented, as demonstrated by the in smaller squares. Another group might be mixed in some respects (e.g. manufacturer and model year) but operate in unique conditions and will need to be sampled separately, as demonstrated by the circles mixed in the smaller triangles.

The following data may be collected for each asset on a 1 Hz basis.

An integrated portable emissions measurement system (iPEMS) may measure NO, NO₂, CO, CO₂, O₂, hydrocarbons, and/or particulate matter/particular number (e.g., with opacity, ionization, and scattering).

An OBM device may measure CO₂, NOx, PM, and NH₃.

An ECU logger may measure 20-40 parameters depending on what is made available by the ECU. Example of commonly requested parameters include fuel use rate, engine load and torque, engine rotation per minute (RPM), intake manifold pressure and temperature, boost pressure, intake air flow rate, air-to-fuel ration (AFR), exhaust gas restriction (EGR), pressure and temperature, exhaust after treatment temperature, diesel exhaust fluid (DEF) dosing, fuel injector pulse, and/or ground speed.

A GPS may measure latitude and longitude, barometric pressure, grade (inclination).

A sensor may measure ambient temperature, pressure, and/or humidity.

Other components may measure need-based measurements such fuel flow rate in supply and return lines using a non-intrusive sensor, temperature of exhaust gas and after treatment, and/or grade using inclinometers.

To operate in extreme weather conditions, a hardened and weatherized protective system may be used to accompany testing of the invention disclosed herein. The protective system was used in this example to allow for uninterrupted testing during inclement weather. The protective system was used during heavy mobile equipment (HME) emissions monitoring at a surface mine. The invention disclosed herein was able to operate in temperatures of −20° C.-50° C. The invention disclosed herein may operate in other temperature conditions, depending on the durability and design of the protective system.

All of the data collected is instantaneous and on a 1 Hz basis. This data can be monitored in near real-time from a remote location. The devices used may be equipped with cellular/LTE connection features.

Emissions and fuel use data may be reported at every second and provide real-time aggregates at user-defined time windows, such as 1-minute, 10-minutes, hourly, and full work-shift (8-hours).

Fuel savings and target CO₂ reduction may be identified by early maintenance issue identification, identifying under-performing assets at an emissions level, enabling operators to understand their impact and to optimize their behaviors influencing emissions reductions, and optimizing haul road performance by identifying areas and roads where changing the road's design could reduce emissions.

Regarding early maintenance issue identification, ECU data and tailpipe data trends for equipment health monitoring and predictive maintenance may be used.

Regarding identifying under-performing assets at an emissions level, instantaneous data may be folded into power demand modes (or work done such as tonne-kms). This allows for the comparison of equipment based on their commercial output. This allows for identification if a larger (and thirstier) equipment might be more efficient than a smaller one. Or, whether an equipment has an optimal value and should not be loaded to maximum capacity.

Regarding enabling operators to understand their impact and to optimize their behaviors influencing emission reductions, real-time graphics may be provided on a dashboard mounted phone or tablet, showing the effect of equipment operation state on fuel use and emissions. As examples, operators can review the impact of extended-idling versus intermittent-idling or no idling; harsh acceleration versus steady-state driving; or warm-up idling versus cold-start. Game modes may be used where the operators may be challenged (against their best performance or other operators) to complete a unit of activity, such as climbing a hill, with the lowest possible impact on fuel use and emissions.

Regarding optimizing haul road performance by identifying areas and roads where changing the road's design could reduce emissions, the engine-power modal analysis methods may be used to identify the impact of road design parameters such as distance, grade, curvature, and surface on equipment emissions.

The system and methods described herein may allow for the understanding of emissions and the reduction of mobile equipment emissions, the improvement of equipment reliability and reduced downtime, the understanding of fuel cost per operation, and for being ready to use power data modeling to estimate what future technology shifts mean for cost-savings or operations management.

Although the present disclosure has been described with respect to one or more particular embodiments and/or examples, it will be understood that other embodiments and/or examples of the present disclosure may be made without departing from the scope of the present disclosure.

1. A system comprising:
an environmental micro-device configured to generate one or more data sets from its environment; and,
a processor configured to:
receive the one or more data sets from the environmental micro-device, wherein the one or more data sets are emissions data, and wherein the one or more data sets includes greenhouse gases and/or criteria pollutants;
normalize the one or more data sets;
bin the one or more data sets based on at least one criteria filter;
determine one or more data groups from the one or more data sets after the binning;
determine a first data centroid for a first portion of the one or more data groups;
add a portion of the one or more data sets to the one or more data groups;
determine a second data centroid for a second portion of the one or more data groups; and
determine a credit representing a decrease between the second data centroid and the first data centroid for the one or more data groups.

2. The system of claim 1, wherein the environmental micro-device comprises at least one sensor, and wherein the sensor is a non-dispersive infrared (NDIR) sensor, a flame-ionization detector (FID) sensor, a diffusion charger sensor, a laser-light scattering sensor, an opacity sensor, an electrochemical sensor, or an optical sensor.

3. The system of claim 1, wherein the environmental micro-device comprises at least one detector, and wherein the detector is a continuous particle counter (CPC) detector and/or a quantum cascade laser infrared spectroscopy (QCL-IR) detector.

4. The system of claim 1, wherein the one or more data sets further comprises one or more of events, weather, location, time, VIN number, or engine type.

5. The system of claim 1, wherein the processor is further configured to verify one or more of the one or more data sets.

6. The system of claim 1, wherein the processor is further configured to store the credit on a distributed ledger or on an encrypted network.

7. The system of claim 1, wherein the binning of one or more data sets is based on one or more of make, model, year, mileage, engine, fuel source, class, intended usage, or weight.

8. A method comprising:
receiving one or more data sets from an environmental micro-device at a processor, wherein the one or more data sets are emissions data, and wherein the one or more data sets includes greenhouse gases and/or criteria pollutants;
normalizing the one or more data sets using the processor;
binning the one or more data sets based on at least one criteria filter using the processor;
determining one or more data groups from the one or more data sets after the binning using the processor;
determining a first data centroid for a first portion of the one or more data groups using the processor;
adding a portion of the one or more data sets to the one or more data groups using the processor;
determining a second data centroid for a second portion of the one or more data groups using the processor; and
determining, using the processor, a credit representing a decrease between the second data centroid and the first data centroid for the one or more data groups.

9. A non-transitory computer readable medium storing a program configured to instruct the processor to execute the method of claim 8.

10. The method of claim 8, further comprising verifying one or more of the one or more of the data sets using the processor.

11. The method of claim 8, further comprising collecting the one or more data sets using the environmental micro-device.

12. The method of claim 8, wherein the one or more data sets further comprise one or more of inputs, outputs, events, weather, location, time, VIN number, or engine type.

13. The method of claim 8, further comprising storing the credit on a distributed ledger or on an encrypted network.

14. The method of claim 8, wherein the binning is based on one or more of make, model, year, mileage, engine, fuel source, class, intended usage, or weight.
